(19) United States
(12) Patent Application Publication
(10) Pub. No.: US 2011/0262519 A1
(43) Pub. Date: Oct. 27, 2011

(54) METHOD OF IMPROVING LENS ROTATION

(76) Inventors: Ross Franklin, Jacksonville, FL (US); Jonathan Hansen, Jacksonville, FL (US); Kurt Moody, St. Augustine, FL (US)

(21) Appl. No.: 13/091,078

(22) Filed: Apr. 20, 2011

Related U.S. Application Data
(60) Provisional application No. 61/327,178, filed on Apr. 23, 2010.

(51) Int. Cl. A61K 9/00; A61K 31/74; A61P 27/10; G02C 7/02

(52) U.S. Cl. 424/429; 351/177; 424/78.04

(57) ABSTRACT

Ophthalmic stabilized lenses have improved rotational properties by treating them with a wetting agent after they are at least initially polymerized.

### CROSS REFERENCE TO RELATED APPLICATION

This application is a non provisional application of U.S. Application No. 61/327,178 filed on Apr. 23, 2010 and claims priority thereto under 35 U.S.C. 121.

### BACKGROUND

Contact lenses have been used commercially to improve vision since the 1950s. The first contact lenses were made of hard materials. Although these lenses are currently used, they are not suitable for all patients due to their poor initial comfort. Later developments in the field gave rise to soft contact lenses, based upon hydrogels, which are extremely popular today. These lenses have higher oxygen permeabilities and such are often more comfortable to wear than contact lenses made of hard materials. However, these new lenses are not without problems.

Contact lenses can be worn by many users for 8 hours to several days in a row without any adverse reactions such as redness, soreness, mucin buildup and symptoms of contact lens related dry eye. However, some users begin to develop these symptoms after only a few hours of use. Many of those contact lens wearers use rewetting solutions to alleviate discomfort associated with these adverse reactions with some success. However the use of these solutions require that users carry extra solutions and this can be inconvenient. For these users a more comfortable contact lens that does not require the use of rewetting solutions would be useful.

Soft toric contact lenses have different designs than soft spherical lenses. The optical zone portion of toric lenses have two powers in them (spherical and cylindrical), created with curvatures generally at right angles to each other. The spherical and cylindrical powers are required to maintain position at the specific angle (cylinder axis) on the eye to provide the required astigmatic vision correction. The mechanical, generally outer zone of toric lenses contains a stabilization system to properly rotate and orient the cylindrical or astigmatic axis into position while being worn on the eye. Rotating the lens to its proper position when the lens moves or when the lens is inserted is important in producing a toric lens. Improvements in this feature are always welcome.

### SUMMARY OF THE INVENTION

The invention is a method of improving the rotation of a stabilized contact lens by treating a polymerized ophthalmic lens with a wetting agent.

In another aspect of the invention, rotational properties of a stabilized contact lens are improved by treating a polymerized contact lens with a wetting agent after initial polymerization.

In yet another aspect of the invention, a stabilized ophthalmic lens is treated with a wetting agent but not prior to the polymerization of the lens.

### DETAILED DESCRIPTION OF THE INVENTION

This invention includes a method of producing stabilized ophthalmic lenses by treating a polymerized stabilized ophthalmic lens with a wetting agent, provided that the ophthalmic lens formulation does not comprise said wetting agent prior to its polymerization.

Toric contact lenses are ordinarily designed to include a mechanism to keep the contact lens rotationally stable on the eye during blinking or while looking around, to maintain the required orientation (cylinder axis) of the spherical and cylindrical powers. These designs may be provided with tiny marks on the lens surface to assist their fitting.

Preferred toric contact lenses feature a stabilization technology that utilizes natural eyelid pressures and specific thickness variations in the lens periphery to establish lens stability on eye. These lenses quickly orient on eye after lens insertion and maintain stability throughout eye movements. The lens works with the eyelid pressures to actively balance the lens in place when the eye is open and quickly re-align the lens if it rotates out of position. Toric lenses or toric multifocal lenses are disclosed in U.S. Pat. Nos. 5,652,638, 5,805,260 and 6,183,082 which are incorporated herein by reference in their entireties.

As used herein, “ophthalmic lens” refers to a device that resides in or on the eye. These devices can provide optical correction or may be cosmetic. Ophthalmic lenses include but are not limited to soft contact lenses, intraocular lenses, overlay lenses, ocular inserts, and optical inserts. The preferred lenses of the invention are soft contact lenses made from silicone elastomers or hydrogels, which include but are not limited to silicone hydrogels, and fluorohydrogels. Soft contact lens formulations are disclosed in U.S. Pat. No. 5,710,302, WO 9421698, EP 406161, JP 2000016905, U.S. Pat. No. 5,998,498, U.S. Pat. No. 6,087,415, U.S. Pat. No. 5,760,100, U.S. Pat. No. 5,776,999, U.S. Pat. No. 5,789,461, U.S. Pat. No. 5,849,811, and U.S. Pat. No. 5,965,631. The foregoing references are hereby incorporated by reference in their entirety. The particularly preferred ophthalmic lenses of the inventions are known by the United States Approved Names of acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, deltafilcon B, dimefilcon A, drooxifilcon A, epsifilcon A, esterifilcon A, etafilcon A, focofilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, silafilcon A, siloxyfilcon A, tefilcon A, tetrafilcon A, trifilcon A, and xylofilcon A. More particularly preferred ophthalmic lenses of the invention are genfilcon A, lenefilcon A, comfilcon, lotrafilcon A, lotraifilcon B, and balafilcon A. The most preferred lenses include etafilcon A, nelfilcon A, hilafilcon, and polymacon.

The term “formulation” refers to the un-polymerized mixture of components used to prepare ophthalmic lenses. These components include but are not limited to monomers, pre-polymers, diluents, catalysts, initiators tints, UV blockers, antibacterial agents, polymerization inhibitors, and the like. These formulations can be polymerized, by thermal, chemical, and light initiated curing techniques described in the foregoing references as well as other references in the ophthalmic lens field. As used herein, the terms “polymerized” or “polymerization” refers to these processes. The preferred methods of polymerization are the light initiated techniques disclosed in U.S. Pat. No. 6,822,016 which is hereby incorporated by reference in its entirety.

As used herein the term “treating” refers to physical methods of contacting the wetting agents and the ophthalmic lens. These methods exclude placing a drop of a solution containing wetting agent into the eye of an ophthalmic lens wearer or placing a drop of such a solution onto an ophthalmic lens prior to insertion of that lens into the eye of a user. Preferably treating refers to physical methods of contacting the wetting agents with the ophthalmic lenses prior to selling or otherwise delivering the ophthalmic lenses to a patient. The ophthalmic lenses may be treated with the wetting agent anytime after they are polymerized. It is preferred that the polymerized ophthalmic lenses be treated with wetting agents at temperature of greater than about 50° C. For example in some processes to manufacture contact lenses, an un-polymerized, or partially polymerized formulation is placed between two mold halves, spincasted, or static casted and polymerized. See, U.S. Pat. Nos. 4,495,313; 4,680,336; 4,889,664, 3,408,429; 3,660,545; 4,113,224; and 4,197,266, all of which are incorporated by reference in their entirety. In the case of hydrogels, the ophthalmic lens formulation is a hardened disc that is subjected to a number of different processing steps including treating the polymerized ophthalmic lens with liquids (such as water, inorganic salts, or organic solutions) to swell, or otherwise equilibrate this polymerized ophthalmic lens prior to enclosing the polymerized ophthalmic lens in its final packaging. Polymerized ophthalmic lenses that have not been swelled or otherwise equilibrated are known as un-hydrated polymerized ophthalmic lenses. The addition of the wetting agent to any of the liquids of this “swelling or “equilibrating” step at room temperature or below is considered “treating” the lenses with wetting agents as contemplated by this invention. In addition, the polymerized un-hydrated ophthalmic lenses may be heated above room temperature with the wetting agent during swelling or equilibrating steps. The preferred temperature range is from about 50° C. for about 15 minutes to about sterilization conditions as described below, more preferably from about 50° C. to about 85° C. for about 5 minutes.

Yet another method of treating is physically contacting polymerized ophthalmic lens (either hydrated or un-hydrated) with a wetting agent at between about room temperature and about 85° C. for about 1 minute to about 72 hours, preferably about 24 to about 72 hours, followed by physically contacting the polymerized ophthalmic lens with a wetting agent at between about 85° C. and 150° C. for about 15 minutes to about one hour.

Many ophthalmic lenses are packaged in individual blister packages, and sealed prior to dispensing the lenses to users. As used herein, these polymerized lenses are referred to as “hydrated polymerized ophthalmic lenses”. Examples of blister packages and sterilization techniques are disclosed in the following references which are hereby incorporated by reference in their entirety, U.S. Pat. Nos. D435,966 S; 4,691,820; 5,467,868; 5,704,468; 5,823,327; 6,050,398, 5,696,686; 6,018,931; 5,577,367; and 5,488,815. This portion of the manufacturing process presents another method of treating the ophthalmic lenses with wetting agents, namely adding wetting agents to packaging solution prior to sealing the package, and subsequently sterilizing the package. This is the preferred method of treating ophthalmic lenses with wetting agents.

Sterilization can take place at different temperatures and periods of time. The preferred sterilization conditions range from about 100° C. for about 8 hours to about 150° C. for about 0.5 minute. More preferred sterilization conditions range from about 115° C. for about 2.5 hours to about 130° C. for about 5.0 minutes. The most preferred sterilization conditions are about 124° C. for about 30 minutes.

The “packaging solutions” that are used in methods of this invention may be water-based solutions. Typical packaging solutions include, without limitation, saline solutions, other buffered solutions, and deionized water. The preferred aqueous solution is deioinized water or saline solution containing salts including, without limitation, sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. Preferably, the packaging solution is a borate buffered or phosphate buffered saline solution or deionized water. The particularly preferred packaging solution contains about 1,850 ppm to about 18,500 ppm sodium borate, most particularly preferred about 3,700 ppm of sodium borate.

As used here, the term “wetting agent” refers polymers having a number average molecular weight of about at least 500, that impart a moist feeling when added to the eyes of contact lens wearers. Examples of preferred wetting agents include but are not limited to poly(meth)acrylamides [i.e. poly N,N-dimethylacrylamide), poly (N-methylacrylamide) poly (acrylamide), poly(N-2-hydroxyethylmethacrylamide), and poly(glucosamineacrylamide)], poly(itaconic acid), hyaluronic acid, xanthan gum, gum Arabic (acacia), starch, polymers of hydroxylalkyl(meth)acrylates [i.e. poly(2-hydroxyethylmethacrylate), poly(2,3-dihydroxypropylmethacrylate, and poly(2-hydroxyethylacrylate)], and polyvinylpyrrolidone.

Additional preferred wetting agents include but are not limited to co-polymers and graft co-polymers of the aforementioned preferred wetting agents, such co-polymers and graft co-polymers include repeating units of hydrophilic or hydrophobic monomers, preferably in amounts of about less than ten percent by weight, more preferably less than about two percent. Such repeating units of hydrophilic or hydrophobic monomers include but are not limited to alkenes, styrenes, cyclic N-vinyl amides, acrylamides, hydroxyalkyl (meth)acrylates, alkyl (meth)acrylates, siloxane substituted acrylates, and siloxane substituted methacrylates. Specific examples of hydrophilic or hydrophobic monomers which may be used to form the above co-polymers and graft co-polymers include but are not limited to ethylene, styrene, N-vinylpyrrolidone, N,N-dimethylacrylamide, 2-hydroxyethylmethyacrylate, methyl methacrylate and butyl methacrylate, methacryloxypropyl tristrimethylsiloxysilane and the like. The preferred repeating units of hydrophilic or hydrophobic monomers are N-vinylpyrrolidone, N,N-dimethylacrylamide, 2-hydroxyethylmethacrylate, methyl methacrylate, and mixtures thereof. Further examples of wetting agents include but are not limited to polymers with carbon backbones and pendant polyethylene glycol chains [i.e. polymers of polyethylene glycol monoomethacrylate] copolymers of ethylene glycol [copolymers with 1,2propyleneglycol, 1,3-propylene glycol, methyleneglycol, and tetramethylene glycol]. The preferred wetting agents are polyvinylpyrrolidone, graft co-polymers and co-polymers of polyvinylpyrrolidone, the particularly preferred wetting agent is polyvinylpyrrolidone. Polyvinylpyrrolidone (“PVP”) is the polymerization product of N-vinylpyrrolidone. PVP is available in a variety of molecular weights from about 500 to about 6,000,000 Daltons. These molecular weights can be expressed in term of K-values, based on kinematic viscosity measurements as described in Encyclopedia of Polymer Science and Engineering, John Wiley & Sons Inc, and will be expressed in these numbers throughout this application. The use of PVP having the following K-values from about K-30 to about K-120 is contemplated by this invention. The more preferred K-values are about K-60 to about K-100, most preferably about K-80 to about K-100. For the treatment of etafilcon A lenses, the particularly preferred K-value of PVP is about K-80 to about K-95, more preferably about K-85 to about K-95, most preferably about K-90.

The wetting agents can be added to the packaging solution at a variety of different concentrations such as about 100 ppm to about 150,000 ppm. For example if the wetting agents are added to packaging solutions containing un-hydrated polymerized ophthalmic lenses, the wetting agents are preferably present at a concentration of about 30,000 ppm to about 150,000 ppm. If the wetting agents are added to packaging solutions containing hydrated polymerized ophthalmic lenses, the wetting agents are preferably present at a concentration of about 100 ppm, to about 3000 ppm, more preferably about 200 ppm to about 1000 ppm, most preferably less than about 500 ppm. For example when etafilcon A lenses are used in this invention and the wetting agent is K-90 PVP, the preferred packaging solution concentration of PVP K-90 is about 250 ppm to about 2,500 ppm, more preferably about 300 to about 500 ppm, most preferably about 350 to about 440 ppm.

When etafilcon A contact lenses are heated with K-90 PVP at a temperature greater than about 120° C. for about 30 minutes at a concentration of about 400 to about 500 ppm, the treated lenses are more comfortable to users than untreated lenses. Further, this particular molecular weight and concentration of PVP does not distort or shift the diameter of the lenses during the treatment cycle or distort the users vision. While not wishing to be bound by any particular mechanism of incorporation, it is known that K-90 PVP is incorporated into the matrix of the lens after it is treated with K-90 PVP. In an etafilcon A contact lens, the preferred amount of incorporated K-90 PVP is about 0.01 mg to about 1.0 mg, more preferred about 0.10 mg to about 0.30 mg, most particularly preferred about 0.10 mg to about 0.20 mg. Lenses that have been treated in this manner are worn by users for up to 12 hours still maintain the incorporated PVP.

Further the invention includes an ocular device comprising, consisting essentially of, or consisting of a polymerized ophthalmic lens wherein said polymerized ophthalmic lens is treated with a wetting agent, provided that the ophthalmic lens formulation does not comprise said wetting agent prior to its polymerization. The terms “ophthalmic lens,” “wetting agent,” “polymerized,” and “formulation” all have their aforementioned meanings and preferred ranges. The term “treated” has the equivalent meaning and preferred ranges as the term treating.

Still further the invention includes an ocular device prepared by treating a polymerized ophthalmic lens with a wetting agent, provided that the ophthalmic lens formulation does not comprise said wetting agent prior to its polymerization. The terms “ophthalmic lens,” “wetting agent,” “polymerized,” “treated” and “formulation” all have their aforementioned meanings and preferred ranges.

The application of the invention is described in further detail by use of the following examples. These examples are not meant to limit the invention, only to illustrate its use. Other modifications that are considered to be within the scope of the invention, and will be apparent to those of the appropriate skill level in view of the foregoing text and following examples.

### EXAMPLES

### Example 1

Cured etafilcon A contact lenses (sold as 1-Day Acuvue® brand contact lenses by Johnson & Johnson Vision Care, Inc.) were equilibrated in deionized water, and packaged in solutions containing PVP in borate buffered saline solution ((1000 mL, sodium chloride 3.55 g, sodium borate 1.85 g, boric acid 9.26 g, and ethylenediamine tetraacetic acid 0.1 g: 5 rinses over 24 hours, 950±μL), sealed with a foil lid stock, and sterilized (121° C., 30 minutes). Before the addition of PVP each solution contained water, 1000 mL, sodium chloride 3.55 g, sodium borate 1.85 g, boric acid, 9.26 g, and ethylenediamine tetraacetic acid 0.1 g. A variety of different weights and concentrations of PVP were used as shown in Table 1, below

The amount of PVP that is incorporated into each lens is determined by removing the lenses from the packaging solution and extracting them with a mixture 1:1 mixture of N,N-dimethylforamide, (DMF) and deionized water (DI). The extracts are evaluated by high performance liquid chromatography (HPLC). Three lenses were used for each evaluation. The results and their standard deviation are presented in Table 1.

TABLE 1

| | | | mg of PVP | |
|---|---|---|---|---|
| Sample # | Type of PVP | Conc. (ppm) | in lens | |
| Control | None | None | none | |
| 1 | K-12 | 3000 | 0.24 | (0.01) |
| 2 | K-12 | 20,000 | 1.02 | (0.01) |
| 3 | K-30 | 1500 | 1.39 | (0.05) |
| 4 | K-30 | 2000 | 1.50 | (0.01) |
| 5 | K-60 | 1000 | 0.56 | (0.00) |
| 6 | K-60 | 1500 | 0.85 | (0.02) |
| 7 | K-60 | 2500 | 1.02 | (0.03) |
| 8 | K-90 | 250 | 0.10 | (0.00) |
| 9 | K-90 | 500 | 0.14 | (0.00) |
| 10 | K-90 | 1000 | 0.2 | (0.01) |
| 11 | K-90 | 2500 | 0.25 | (0.02) |
| 12 | K-120 | 500 | 0.07 | (0.00) |

### Example 2

Samples of treated etafilcon A lenses were prepared via the treatment and sterilization methods of Example 1 from K-12, K-30, K-60, K-90, and K-120 PVP at concentrations of 0.30%, 1.65%, and 3.00%. After sterilization, the diameter of the lenses was, compared to an untreated lens and evaluated to determine if the process changed those diameters. The results, Table, plot the change in diameter vs the type of PVP at a particular concentration. This data shows that K-12, K-90, and K-120 have a minimal effect on the diameter of the lenses.

### Example 3

Several etafilcon A lenses were treated with K-90 PVP at a concentration of 500 ppm and sterilized according to the methods of Example 1. The lenses were stored in their packages for approximately 28 days at room temperature and were then measured for diameter, base curve, sphere power, and center thickness. Thereafter, lenses were heated at 55° C. for one month. The diameter, base curve, sphere power, and center thickness of the lenses was measured and the results were evaluated against an untreated lens and data is presented in Table 2. This data illustrates that the parameters of lenses treated with K-90 PVP are not significantly affected by time at elevated temperature.

TABLE 2

| | | | Change from |
|---|---|---|---|
| | | | Baseline of |
| | | | Sample after one |
| | | | month storage at |
| | Baseline | | 55° C. |
| Diameter (mm) | 14.37 | (0.02) | 0.02 |
| Base curve (mm) | 8.90 | (0.03) | −0.01 |
| Power (diopter) | −0.75 | (0.05) | 0.00 |
| Center Thickness | 0.127 | (0.005) | 0.002 |

(mm)

### Example 4

Etafilcon-A lenses treated with PVP K-90 at a concentration of 440 ppm and sterilized (124° C., approximately 18 minutes) were sampled from manufacturing lines and measured for diameter, base curve, sphere power, and center thickness and compared to similar measurements made on untreated 1-Day Acuvue® brand lenses. The data presented in Table 3 illustrates that K-90 PVP does not significantly affect these parameters.

TABLE 3

| | Treated | | Untreated | |
|---|---|---|---|---|
| Diameter (mm) | 14.24 | (0.04) | 14.18 | (0.04) |
| Base curve (mm) | 8.94 | (0.03) | 8.94 | (0.04) |
| Sphere Power Deviation from | −0.01 | (0.04) | −0.02 | (0.04) |
| Target (diopter) | | | | |
| Center Thickness Deviation | 0.000 | (0.004) | 0.002 | (0.005) |
| from Target (mm) | | | | |

### Example 5

Etafilcon A lenses were prepared according to Example 1 at the concentrations of Table 1. The treated lenses were clinically evaluated in a double-masked studies of between 9 and 50 patients. The patients wore the lenses in both eyes for 3-4 days with overnight removal and daily replacement, and wore untreated 1-Day Acuvue® brand contact lenses for 3-4 days with overnight removal and daily replacement as a control. Patients were not allowed to use rewetting drops with either type of lens. Patients were asked to rate the lens using a questionnaire. All patients were asked a series of questions relating to overall preference, comfort preference, end of day preference, and dryness. In their answers they were asked to distinguish if they preferred the treated lens, the 1-Day control lens, both lenses or neither lens. The results are shown in Tables 4 and 5. The numbers in the columns represent the percentage of patients that positively responded to each of the four options. The “n” number represents the number of patients for a particular sample type. “DNT” means did not test and n/a means non applicable. The numbers illustrate that lenses treated with K-90 PVP at a concentration of about 500 ppm have good clinical comfort on the eye. The sample # refers to the sample numbers in Table 1.

TABLE 4

| | | Overall Preference, % | | | | Comfort Preference, % | | | |
|---|---|---|---|---|---|---|---|---|---|
| | | PVP | | | | PVP | | | |
| Sample # | n | treated | 1-Day | Both | Neither | treated | 1-Day | Both | Neither |
| 1 | 9 | 67 | 22 | 11 | 0 | 67 | 22 | 11 | 0 |
| 2 | 37 | 27 | 49 | 22 | 3 | 30 | 46 | 19 | 5 |
| 3 | 41 | 34 | 49 | 15 | 2 | 27 | 56 | 12 | 5 |
| 4 | 10 | 30 | 20 | 50 | 0 | 30 | 40 | 30 | 0 |
| 5 | 41 | 27 | 61 | 10 | 2 | 22 | 49 | 29 | 0 |
| 6 | 42 | 33 | 33 | 33 | 0 | 33 | 29 | 38 | 0 |
| 7 | 37 | 51 | 27 | 19 | 3 | 49 | 11 | 38 | 3 |
| 8 | 41 | 27 | 37 | 32 | 5 | 24 | 34 | 37 | 5 |
| 9 | 48 | 33 | 27 | 40 | 0 | 33 | 23 | 44 | 0 |
| 10 | 45 | 18 | 27 | 51 | 4 | 16 | 20 | 58 | 7 |

TABLE 5

| | | Dryness Preference % | | | | End of Day Preference % | | | |
|---|---|---|---|---|---|---|---|---|---|
| | | PVP | | | | PVP | | | |
| Sample # | n | treated | 1-Day | Both | Neither | treated | 1-Day | Both | Neither |
| 1 | 9 | 33 | 33 | 11 | 0 | 56 | 22 | 44 | 0 |
| 2 | 37 | 24 | 43 | 22 | 8 | 27 | 43 | 27 | 5 |
| 3 | 41 | 32 | 51 | 17 | 2 | 29 | 49 | 17 | 2 |
| 4 | 10 | 20 | 40 | 30 | 10 | 20 | 10 | 60 | 10 |
| 5 | 41 | 20 | 46 | 32 | 2 | 20 | 41 | 37 | 2 |
| 6 | 31 | 42 | 24 | 38 | 0 | 38 | 35 | 16 | 6 |
| 7 | 42 | 36 | 19 | 38 | 3 | 41 | 24 | 40 | 0 |
| 8 | 41 | 27 | 22 | 49 | 7 | 22 | 24 | 41 | 7 |
| 9 | 48 | 38 | 21 | 46 | 0 | 33 | 19 | 44 | 0 |
| 10 | 45 | 24 | 20 | 58 | 4 | 18 | 20 | 51 | 4 |

### Example 6

An etafilcon A contact lens was treated with 500 ppm of K-90 PVP using the methods of Example 1. The treated lenses were briefly rinsed with phosphate buffered saline solution and rinsed lenses were placed in the well of a cell culture cluster container (Cellgrow XL) that mimics the dimensions of a human eye. See, Farris R L, Tear Analysis in Contact Lens Wears, Tr. Am. Opth. Soc. Vol. LXXXIII, 1985. Four hundred microliters of phosphate buffered saline solution (KH₂PO₄ 0.20 g/L, KCl 0.20 g/L, NaCl 8.0 g/L, Na₂HPO₄ [anhydrous] 1.15 g/L) was added to each container. The wells were covered and the container was stored in an oven at 35° C.

Three lenses were removed from the oven at various times and analyzed by HPLC to determine whether PVP was released into the phosphate buffered saline solution. The average results are presented in Table 6. The limit of quantification for PVP is 20 ppm. The test did not detect any PVP in the analyzed samples. This data shows that PVP is not released at levels greater than 20 ppm.

TABLE 6

| | PVP |
|---|---|
| Time | Released |
| 30 min. | <20 ppm |
| 1 hr. | <20 ppm |
| 2 hr. | <20 ppm |
| 4 hr. | <20 ppm |
| 8 hr. | <20 ppm |
| 16 hr. | <20 ppm |
| 24 hr | <20 ppm |

### Example 7

Contact lens for astigmatic patients having a known design with the following input design parameters were made according to the method set out in Example 1. The following lens parameters were obtained:
Sphere power: −9.00 D-+6.00 D
Cylinder Power: −0.75 D-−2.25 D
Cylinder Axis: 10 deg-180 deg
Lens diameter: 14.50 mm
Front optical zone diameter of 8.50 mm
Back optical zone diameter of 11.35 mm
Lens base curve: 8.55 mm

The thickness profile of the lenses is non-rotationally symmetrical in the peripheral zone. The stabilization zone is an extra thick zone added to the thickness profile of the lenses.

### Example 8

### Prophetic

Thirty astigmatic patients are fitted with lenses made according to Example 6 using their current corrective prescriptions. An optometrist removes a stabilized lens from a fresh package and inserts the lens onto the patient's eye so that the axis is 90° from its correct position on the eye. Both the time and number of blinks that it takes the lens to rotate to its correct position on-eye is recorded. The procedure is repeated using a hydrogel lens (not made according to the inventive method). On average, lenses made according to the inventive method rotated to within 10° of their correct positions within 20 seconds (and 4-5 blinks). On average, lenses not made according to the inventive method required more than 30 seconds (and 7-8 blinks) to rotate to within 10° of their correct positions.

### Visual Acuity and Gaze Analysis (Prophetic)

This is a 14 subject, one visit, and randomized, unmasked non-dispensing study. This study is in two sections; the first part studies the effect of gravity on toric lens rotation and subsequent change in visual acuity (VA). The second part will look at the rotation of the lens in response to change in gaze direction. In the first part, subjects wear, in random succession, four lenses in each eye: lens according to the invention, Purevision Toric (PVT), Air Optix Toric (AOT) and Proclear Toric (PCT). After a settling period of 15 minutes the visual acuity is tested in both the upright and recumbent positions and orientation of the lens is photographed in the recumbent position. The procedure is repeated in the second part of the study but a continuous recording of the subjects looking between their primary gaze position and each of the eight cardinal directions of gaze is taken. Lens rotation is captured using the Sony 3CCD exwaveHAD video recorder and Broadway computer software (Data Translation Inc, 1996/1997). Lens orientation position measurements are undertaken from the video recording by using Ulead Video Studio 11 (2007, Corel Corporation) and Pixel Port v 1.1.

Lenses according to the invention are found to rotate significantly less from their settled orientation with subjects in the recumbent position than the three other lenses. The mean final orientation position of the subjects in the recumbent position is 11.0° infero temporally with lenses according to the invention, compared with; PVT 28.7° (P<0.0001), AOT 26.5° (P=0.001) and PCT 29.1° (P<0.0001).

Upright visual acuity is assessed first and once the lens re-orientates due to gravity, with the subject in the recumbent position, visual acuity is assessed again. Mean VA in the recumbent position is significantly worse for two of the three lenses not according to the invention, when compared with the lens according to the invention. Mean VA in the recumbent position is 0.00 log MAR for the lens according to the invention compared with +0.17 log MAR for PVT (P=0.01) and +0.11 log MAR for PCT (P=0.04). The mean visual acuity for AOT in the recumbent position is +0.05 log MAR. High contrast visual acuity is measured in one eye only for each subject using a Bailey-Lovie test chart (0.02 log MAR=1 letter). The difference in VA from the upright to the recumbent position decreases by 0.03 log MAR for the lens according to the invention. This result is significantly worse for PVT lenses, with the mean VA decreasing by 0.17 log MAR(P=0.04). The mean visual acuity with AOT lenses decreased by 0.04 log MAR when changing from the upright to recumbent position and PCT decreases by 0.10 log MAR (1 line).

For the gaze analysis subjects are positioned at the slit-lamp in front of a chart with targets positioned 45° apart in eight of the cardinal directions of gaze (away from the primary position) at an angle of approximately 40°-45° from the primary position of gaze. Subjects are asked to blink naturally while looking in the primary gaze direction, after four blinks they were asked to look at the 12 o'clock position for a period of four blinks before returning to the primary direction of gaze. If the lens appears not to have rotated then four blinks are counted before they look in the second direction (moving anti-clockwise) for four blinks and so on until the cover all eight directions of gaze. If the lens shows re-orientation after looking into one of the off-axis directions of gaze, the subject is asked to continue blinking normally while looking in the primary position until the lens appears to have settled back into its original position. Subjects are allowed a short practice session prior to beginning filming. Lens movements is video recorded continuously. Upon completion, the lens is removed and the next pair inserted (as per the randomization log) and allowed to settle for 15 minutes.

The PVT and PCT lenses show the greatest amount of orientation change following superior and supero-temporal versions. There is a significant difference between CT and PVT when looking at the mean change in orientation in the nasal direction following the superior gaze direction (5.6° Vs. 0.7°, PVT and CT respectively, P=0.03). When looking at the mean absolute change in orientation, PCT is shown to re-orientate significantly more than lens according to the invention (6.5° Vs. 3.3°, P=0.04). Following the supero-temporal gaze direction PVT shows significantly greater mean absolute change in orientation compared with lenses according to the invention (9.4° Vs. 6.3°, P=0.04).

All three lenses that are not according to the invention exhibit a significantly greater change in orientation than lenses according to the invention following the inferior-nasal gaze direction. Lenses according the invention show a mean absolute change in orientation following this gaze direction of 3.0° compared with; PVT (9.0°, P=0.008), AOT (5.9°, P=0.005) and PCT (9.5°, P=0.006). There are no significant differences in change in lens orientation following temporal, nasal, superior-nasal, infero-temporal or inferior gaze directions

We claim:

1. A method of improving the rotational properties of a stabilized ophthalmic lenses comprising, treating a polymerized stabilized ophthalmic lens with a wetting agent, provided that the ophthalmic lens formulation does not comprise said wetting agent prior to its polymerization.

2. The method of claim 1 wherein treating comprises heating the polymerized ophthalmic lens in a packaging solution.

3. The method of claim 2 wherein the polymerized ophthalmic lens is heated to a temperature of at least about greater than 50° C. to about 150° C.

4. The method of claim 1 wherein the packaging solution comprises deionized water, or saline solution.

5. The method of claim 1 wherein the packaging solution comprises about 1870 ppm to about 18,700 ppm sodium borate

6. The method of claim 1 wherein the packaging solution comprises about 3700 ppm sodium borate.

7. The method of claim 1 wherein the packaging solution comprises about 2000 ppm to about 5000 ppm sodium borate.

8. The method of claim 1 wherein the wetting agent is selected from the group consisting of poly(meth)acrylamides, poly(itaconic acid), hyaluronic acid, xanthan gum, gum Arabic, starch, polymers of hydroxylalkyl(meth)acrylates, and polyvinylpyrrolidone.

9. The method of claim 1 wherein the wetting agent is selected from the group consisting of polyvinylpyrrolidone, graft co-polymers of polyvinylpyrrolidone, and co-polymers of polyvinylpyrrolidone.

10. The method of claim 1 wherein the wetting agent is polyvinylpyrrolidone.

11. The method of claim 10 wherein the K-value of the polyvinylpyrrolidone is about K-60 to about K-120.

12. The method of claim 10 wherein the K-value of the polyvinylpyrrolidone is about K-85 to about K-95.

13. The method of claim 10 wherein the K-value of polyvinylpyrrolidone is about K-90.

14. The method of claim 1 wherein treating comprises heating the polymerized ophthalmic lens in a packaging solution comprising polyvinylpyrrolidone having a K-value of about K-85 to about K-95 at a temperature of greater than about 80° C.

15. The method of claim 1 wherein treating comprises heating the polymerized ophthalmic lens in a packaging solution comprising polyvinylpyrrolidone having a K-value of about K-85 to about K-95 at a temperature of greater than about 120° C.

16. The method of claim 2 wherein the wetting agent is polyvinylpyrrolidone and the concentration of polyvinylpyrrolidone in the packaging solution is about 250 ppm to about 2500 ppm.

17. The method of claim 2 wherein the concentration of wetting agent in the packaging solution is about 100 ppm to about 3000 ppm.

18. The method of claim 1 wherein the polymerized ophthalmic lens is heated at about 124° C. for about 18 minutes and with polyvinylpyrrolidone having a K-value of about K-90 at a concentration of about 400 to about 440 ppm.

19. The method of claim 1 wherein the polymerized ophthalmic lens is heated at about 121° C. for about 30 minutes and with polyvinylpyrrolidone having a K-value of about K-90 at a concentration of about 300 to about 400 ppm.

20. The method of claim 2 wherein the treating step is conducted in an individual sealed contact lens package.

21. The method of claim 17 wherein the treating step is conducted in an individual sealed contact lens package.

22. The method of claim 18 wherein the treating step is conducted in an individual sealed contact lens package.

23. The method of claim 1 wherein the ophthalmic lens is selected from the group consisting of acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, deltafilcon B, dimefilcon A, drooxifilcon A, epsifilcon A, esterifilcon A, etafilcon A, focofilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, silafilcon A, siloxyfilcon A, tefilcon A, tetrafilcon A, trifilcon A, and xylofilcon A.

24. The method of claim 2 wherein the ophthalmic lens is selected from the group consisting of acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, deltafilcon B, dimefilcon A, drooxifilcon A, epsifilcon A, esterifilcon A, etafilcon A, focofilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, silafilcon A, siloxyfilcon A, tefilcon A, tetrafilcon A, trifilcon A, and xylofilcon A.

25. The method of claim 14 wherein the ophthalmic lens is selected from the group consisting of acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, deltafilcon B, dimefilcon A, drooxifilcon A, epsifilcon A, esterifilcon A, etafilcon A, focofilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, silafilcon A, siloxyfilcon A, tefilcon A, tetrafilcon A, trifilcon A, and xylofilcon A.

26. The method of claim 2 wherein the ophthalmic lens is selected from the group consisting of genfilcon A, lenefilcon A, lotrafilcon A, lotrafilcon B, balafilcon A, comfilcon, etafilcon A, nelfilcon A, hilafilcon, and polymacon.

27. The method of claim 14 wherein the ophthalmic lens is selected from the group consisting of genfilcon A, lenefilcon A, lotrafilcon A, lotrafilcon B, balafilcon A, comfilcon, etafilcon A, nelfilcon A, hilafilcon, and polymacon.

28. The method of claim 2 wherein the ophthalmic lens is selected from the group consisting of etafilcon A, nelfilcon A, hilafilcon, and polymacon.

29. The method of claim 14 wherein the ophthalmic lens is selected from the group consisting of etafilcon A, nelfilcon A, hilafilcon, and polymacon.

30. The method of claim 2 wherein the ophthalmic lens is selected from the group consisting of etafilcon A.

31. The method of claim 14 wherein the ophthalmic lens is selected from the group consisting of etafilcon A.

32. The method of claim 18 wherein the ophthalmic lens is an etafilcon A contact lens.

33. The method of claim 1 wherein the polymerized ophthalmic lens is an un-hydrated polymerized ophthalmic lens.

34. The method of claim 33 wherein treating comprises contacting the un-hydrated polymerized ophthalmic lens with a packaging solution comprises about 1870 ppm to about 18,700 ppm sodium borate.

35. The method of claim 33 wherein the treating further comprises heating the un-hydrated polymerized ophthalmic lens and packaging solution to a temperature of at least about 50° C. to about 100° C.

36. The method of claim 33 wherein the treating further comprises maintaining the un-hydrated polymerized ophthalmic lens and packaging solution at a temperature of at least about 10° C. to about room temperature.

37. The method of claim 34 wherein the packaging solution further comprises deionized water, or saline solution.

38. The method of claim 33 wherein the wetting agent is selected from the group consisting of poly(meth)acrylamides, poly(itaconic acid), hyaluronic acid, xanthan gum, gum Arabic, starch, polymers of hydroxylalkyl(meth)acrylates, and polyvinylpyrrolidone.

39. The method of claim 33 wherein the wetting agent is selected from the group consisting of polyvinylpyrrolidone, graft co-polymers of polyvinylpyrrolidone, and co-polymers of polyvinylpyrrolidone.

40. The method of claim 33 wherein the wetting agent is polyvinylpyrrolidone.

41. The method of claim 33 wherein the K-value of the polyvinylpyrrolidone is about K-60 to about K-120.

42. The method of claim 33 wherein the K-value of the polyvinylpyrrolidone is about K-60 to about K-90.

43. The method of claim 40 wherein the polyvinylpyrrolidone is present at a concentration of about 30,000 ppm to about 150,000 ppm.

44. The method of claim 34, further comprising a second step of heating the un-hydrated polymerized ophthalmic lens of claim 34 with a second portion of packaging solution comprising a second wetting agent.

45. The method of claim 44 wherein the wetting agent is polyvinylpyrrolidone having a K-value of about K-60 and the second wetting agent is polyvinylpyrrolidone having a K-value of about K-90.

46. The method of claim 44 wherein the wetting agent is polyvinylpyrrolidone having a K-value of about K-90 and the second wetting agent is polyvinylpyrrolidone having a K-value of about K-90.

47. The method of claim 45 wherein the concentration of K₆₀ is between about 30,000 ppm and 150,000 about ppm and the concentration of K₉₀ is between about 100 ppm and about 500 ppm.

48. An ocular device comprising a polymerized stabilized ophthalmic lens wherein said polymerized stabilized ophthalmic lens is treated with a wetting agent, provided that the ophthalmic lens formulation does not comprise said wetting agent prior to its polymerization.

49. The device of claim 48 wherein the wetting agent is selected from the group consisting of poly(meth)acrylamides, poly(itaconic acid), hyaluronic acid, xanthan gum, gum Arabic, starch, polymers of hydroxylalkyl(meth)acrylates, and polyvinylpyrrolidone.

50. The device of claim 48 wherein the wetting agent is polyvinylpyrrolidone, graft co-polymers of polyvinylpyrrolidone, and co-polymers of polyvinylpyrrolidone.

51. The device of claim 48 wherein the wetting agent is polyvinylpyrrolidone.

52. The device of claim 48 comprising about 0.01 mg to about 1.0 mg polyvinylpyrrolidone.

53. The device of claim 48 comprising about 0.10 mg to about 0.44 mg of polyvinylpyrrolidone.

54. The device of claim 48 wherein said device does not distort the user's vision.

55. The device of claim 48 comprising about 0.01 mg to about 1.0 mg of a wetting agent selected from the group consisting of poly(meth)acrylamides, poly(itaconic acid), hyaluronic acid, xanthan gum, gum Arabic, starch, polymers of hydroxylalkyl(meth)acrylates, and polyvinylpyrrolidone.

56. The device of claim 48 comprising about 0.10 mg to about 0.44 mg of poly(meth)acrylamides, poly(itaconic acid), hyaluronic acid, xanthan gum, gum Arabic, starch, polymers of hydroxylalkyl(meth)acrylates, and polyvinylpyrrolidone.

57. The device of claim 48 wherein said device does not distort the user's vision.

58. The device of claim 48 wherein said wetting agent remains in the ophthalmic lens after about 6 hours to about 24 hours of wear by a user.

59. An ocular device prepared by treating a polymerized ophthalmic lens with a wetting agent, provided that the ophthalmic lens formulation does not comprise said wetting agent prior to its polymerizaton.

60. The device of claim 59 wherein treating comprises heating the polymerized ophthalmic lens in a packaging solution to a temperature of about greater than 50° C. to about 150° C.

61. The device of claim 59, wherein the wetting agent is selected from the group consisting of polyvinylpyrrolidone, graft co-polymers of polyvinylpyrrolidone, and co-polymers of polyvinylpyrrolidone.

62. The device of claim 59 wherein the wetting agent is polyvinylpyrrolidone having a K-value of about K-60 to about K-90.

63. The device claim 59 wherein the ophthalmic lens is selected from the group consisting of acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, deltafilcon B, dimefilcon A, drooxifilcon A, epsifilcon A, esterifilcon A, etafilcon A, focofilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, silafilcon A, siloxyfilcon A, tefilcon A, tetrafilcon A, trifilcon A, and xylofilcon A.

64. The device of claim 59 wherein the ophthalmic lens is selected from the group consisting of genfilcon A, lenefilcon A, lotrafilcon A, lotrafilcon B, balifilcon A, comfilcon, etafilcon A, nelfilcon A, hilafilcon, and polymacon.

65. The device of claim 59 wherein the ophthalmic lens is etafilcon A.
